(19) United States
(12) Patent Application Publication
(10) Pub. No.: US 2021/0361696 A1
(43) Pub. Date: Nov. 25, 2021

(54) BIOLOGICALLY ACTIVE COMBINATORIAL POLYSACCHARIDE DERIVATIVES

(71) Applicants: Boris Farber, Brooklyn, NY (US); Sof'ya Farber, Brooklyn, NY (US); ARTUR VIKTOROVICH MARTYNOV, Kharkov (UA)

(72) Inventors: Boris Farber, Brooklyn, NY (US); Sof'ya Farber, Brooklyn, NY (US); ARTUR VIKTOROVICH MARTYNOV, Kharkov (UA)

(21) Appl. No.: 16/771,573

(22) PCT Filed: Jun. 16, 2017
(86) PCT No.: PCT/RU2017/000422

§ 371 Date: Jun. 10, 2020

(51) Int. Cl. A61K 31/727; A61K 31/717; A61K 31/722; A61K 31/718; A61P 37/02; A61P 31/12; A61P 17/02; A61P 7/04

(52) U.S. Cl. CPC A61K 31/727; A61K 31/717; A61K 31/722; A61P 7/04; A61P 37/02; A61P 31/12; A61P 17/02; A61K 31/718

(57) ABSTRACT

The invention related to organic and bioorganic combinatorial chemistry, namely, to new combinatorial libraries of polysaccharide derivatives and supramolecular structures based on them, which, when used without separation into separate components, have high biological activity.

The essence is a combinatorial library and a supramolecular structure based on it from biologically active derivatives of polysaccharides, as well as pharmaceutical compositions based on them with a hemostatic, wound healing, antiviral and immunomodulating action, containing as an active substance an undivided whole combinatorial mixture of substituted glucopyranose polymer derivatives, obtained simultaneous combinatorial modification of a polysaccharide with at least two covalent modifier in the synthesis, a combinatorial mixture with the maximum number of combinations of modified polysaccharide derivatives is formed, and as a biologically active substance, a whole combinatorial mixture of polysaccharide derivatives in the form of a supramolecular structure without separation into individual components is used to obtain a pharmaceutical composition.

### TECHNICAL FIELD

The invention relates to organic and bioorganic combinatorial chemistry, in particular, to new combinatorial libraries of derivatives of polysaccharides and supramolecular structures based on them, which, when used without separation into individual components, have high biological activity.

### PREVIOUS LEVEL OF ART

### Terminology

A pharmaceutical composition with a hemostatic effect is a mixture of several substances, including several biological active hemostatic and wound healing, as well as several auxiliary and formative substances in the form of a sterile powder for topical use, which can also be used to accelerate wound healing, to stop bleeding due to capillary, venous, arterial bleeding. Hemostatic effect of pharmaceutical composition due to the ability to quickly swell in the wound and cause tamponade in the bleeding site. An analogue of the proposed drug is the drug “Celox” (Celox), which can absorb blood from a wound and turn into a gel, thereby closing open bleeding.

The carboxylated derivative of the glucopyranose polymer [C₆H₇O₂ (OH) 3-x (O—R—COOH) x]n, where x=0.05-3, R=—CH2-; —CO—CH₂—CH₂—; —CO—CH═CH— and others) is a derivative of the glucopyranose polymer (FIG. 1), in which carboxylation of glucose residues is carried out by introducing a carboxyl group (—COOH). This is followed by alkylation or acylation of hydroxyl residues of glucose (glucopyranose). In this process glucopyranose monomers are interconnected by 1,4-glycosidic bonds. Modification of alcohol hydroxyls of glucopyranose can be carried out with a different degree of substitution (x=0.08-3) by carboxylation (carboxycellulose or carboxy starch).

For the carboxylation of polysaccharides, maleic, succinic, phthalic and other anhydrides of di- and poly-carboxylic acids can be used, and for alkylation will be used substances such as monochloroacetic acid (while carboxymethyl cellulose or carboxymethyl starch are formed).

Cellulose—is one of the most common natural polymers of a polysaccharide nature. Polysaccharide nature refers to the main component of the cell walls of plants, which determine the mechanical strength and elasticity of plant tissues. Cellulose macromolecules are constructed from D-glucose units linked by 1,4-beta-glycosidic bonds into linear unbranched chains. The average degree of polymerization of cellulose (the number of glycosidic residues) varies widely—from several hundred (for viscose fiber pulp it is 300-500) to 10-14 thousand (for cotton fiber and bast fibers). Cellulose has a complex supramolecular structure. The primary element is a microfibril, consisting of several hundred macromolecules and having the form of a spiral (thickness 35-100, length 500-600 nm and above).

Carboxymethyl cellulose—(CMC, cellulose glycolic acid, [C₆H₇O₂ (OH) 3-x (OCH₂COOH) x] n, where x=0.08-1.5) is a cellulose derivative in which cellulose is carboxylated by introducing a carboxylmethyl group (—CH₂—COOH) through a covalent bond with hydroxyl groups of glucose monomers. Na-carboxymethyl cellulose is used as a plasticizer, thickener, and resorbent. As a thickening agent, it is a part of toothpaste, food products, cosmetics, medicines.

Carboxypropyl cellulose—is a CPC, a cellulose derivative in which carboxylation of cellulose is carried out by introducing a carboxypropyl group (—CH₂—CH₂—CH₂—COOH) through a covalent bond with the hydroxyl groups of glucose monomers. It is used in tablet coatings and as a prolonging excipient in the production of various dosage forms of drugs.

Carboxymethyl starch—(CMS) is a modified starch, ether starch, and water-soluble anionic polymer. It is odorless, non-toxic, and linear in form with a degree of substitution of more than 0.2 or more soluble in water. Formula: K₆H₇O₂(OH)₂OCH₂COONa and it can be used as an emulsifier, thickener, dispersant, stabilizer, adhesive, or film-forming agent. It is widely used in oil, textile, chemical, tobacco, paper, construction, food, pharmaceutical and other industries. Commonly used sodium salt, also known as (CMS-Na). It is a white or yellow powder, without taste and odor, non-toxic, and easily absorbs moisture. Additionally, it is soluble in alcohol, ether, chloroform and other organic solvents.

Combinatorial polysaccharide derivative (CPP) is a polysaccharide derivative obtained according to the examples of the present invention containing an indivisible supramolecular mixture of different combinatorial modified polysaccharides.

### Cellulose Derivatives

Due to the presence of hydroxyl groups in elementary macromolecules, cellulose is easily esterified and alkylated; these reactions are widely used in industry to produce cellulose ethers and esters. Many cellulose derivatives are able to form elastic films, which determines their use in the production of various medicines.

The preparation processes uses high-quality reverse osmosis water (including for medical purposes), produces several grades of cellulose acetate membranes (MGA series), which have a selectivity for sodium chloride from 70 to 90%. Ultra cellulose membranes (pore sizes from 5 to 50 nm) based on cellulose acetate are used for the purification and concentration of proteins, enzymes, and antibiotics. Microfiltration membranes (pore sizes from 100 to 1000 nm) are used in microbiological, biological and physical-chemical analyzes, for cleaning microorganism solutions of drugs, sterilizing filtration, electrophoretic separation of blood serum proteins and other high molecular weight compounds.

The use of cellulose acetate (AC) for microencapsulation of low and high molecular weight drugs becomes promising. Typically, polymer microcapsules are of the order of tens or hundreds of microns, and the membrane thickness is hundredths or tenths of a micron. Microencapsulated drugs (microcapsule size less than 20 microns) are introduced into ointment bases, used to prepare syrups and other liquid dosage forms. Microencapsulation is used in the preparation of injectable mixtures in the form of suspensions of microcapsules for intramuscular and subcutaneous administration with controlled release.

The use of cellulose acetate for microencapsulation of drugs made it possible to obtain microcapsules with a release rate of the drug, depending on the size of the microcapsule.

Cellulose acetates are used as a polymer permeable membrane when immobilizing enzymes (glucooxidase, invertase, esterase, etc.), as well as polyenzyme systems (glucose oxidase and catalase, glucose oxidase and peroxidase). Using cellulose triacetate, fibrous immobilized enzymes were obtained.

In the production of tablets, AC is used to create a film that protects the medicinal substance from the effects of the external environment. It is also used as a binding and granulating substance.

Water-soluble AC is used to coat tablets of various drugs (glucose, terpinghydrate, aspen, ascofen, amidopyrine, etc.) and serves as a protective coating, providing a prolonged action of the drug. The film-forming properties of methyl cellulose (MC) allow it to be used as a protective coating for medicinal substances for either enteral or topical use. By dissolving or suspending drugs for various purposes in a solution of MC with a polymer concentration of up to 60%, single-dose drugs in the form of films with a thickness of 0.05-1 mm are obtained.

From methylhydroxypropyl cellulose (MHPC), film coatings are prepared for gastro-soluble solid dosage forms.

Phthalyl, acetylphthalyl, acetylsuccinyl—cellulose derivatives (manufactured by Japanese companies) are widely used in the manufacture of medicines. The shell of tablets of such polymers does not dissolve in the stomach (pH=1.4) and protects the drug from the harmful effects of contents in the stomach. Once in the intestines (pH=6.7-7.4), the tablet shell dissolves, which allows the drug to quickly absorb into the blood.

The sodium salt of carboxymethyl cellulose (Na-CMC) can be used as a protective shell for suppositories intended for use in places with a hot climate. Tablets with good appearance and satisfactory strength and disintegration characteristics in the body are usually obtained using (1-8)% Na-CMC solutions. CMC aluminum salt in the form of a 1-5% aqueous solution is used for the manufacture of rapidly disintegrating vaginal tablets.

Films of Na-CMC have a pronounced stimulating effect on reparative processes in infected skin wounds, accelerate the formation and maturation of granulation tissue, and actively influence fibrillogenesis processes. An effective tool for the treatment of long-term non-healing radiation burns is an ointment, which is a Na-CMC gel containing an anti-inflammatory substance-fodomos. Ointments based on Na-CMC are used as light-protective coating and cooling pastes. Bactericidal fluids containing Na-CMC form water-washable films and can be used to treat external wounds.

Pure methyl cellulose hydrogels are used as a drying ointment or wet dressing, as well as protective ointments when working with organic solvents and aggressive media. For the treatment of skin diseases, burns and local anesthesia mainly used MC-based ointments containing anesthetics, antibiotics, silver salts, mercury, zinc, etc.

For the treatment of wounds and burns, the use of monocarboxyl cellulose (MCC) in the form of a sedimentation-resistant aqueous suspension, which forms a film on a drying wound surface, has been proposed. This form of MCC can be considered as a biomaterial that combines the properties of a wound cover and a therapeutic agent that stimulates healing. Acceleration of healing of burns with the help of MCC is 35%. The range of therapeutic effects of the suspension can be significantly expanded by introducing biologically active substances into its composition. Since the pharmacological effect of many drugs is determined by the presence of the corresponding chemical groups in their composition, a new approach to the synthesis of drug polymers using the chemical properties of cellulose derivatives has been implemented. The prescribed character of the pharmacological action is given to the polymer by introducing the corresponding chemical groups into the polymer chain.

The prolonged action of drugs can also be achieved by attaching them to the polymer matrix with a relatively labile covalent bond, in particular, ester or amide. To obtain such derivatives, the drug fixation reaction is carried out with carboxymethyl cellulose chloride. Previously, combinatorial derivatives of polysaccharides were not obtained, no double modification was performed, and the biological properties of such compounds are not known.

### Non-Starch Polysaccharides

From a chemical point of view, carbohydrates are divided into “sugars” (mono- and disaccharides), oligosaccharides and polysaccharides. Oligo- and polysaccharides include compounds whose molecules are built from monosaccharide residues connected by O-glycosidic bonds. The distinction between oligosaccharides and polysaccharides cannot be made strictly, but from a methodological point of view it is advisable to consider compounds containing up to 8-10 monosaccharide units as oligosaccharides, and consider higher molecular weight sugars as polysaccharides. The main components of dietary fiber are polysaccharides, which form both linear and branched chains. An important role in determining the physical properties and ability of polysaccharides to form associations with other polysaccharides and proteins is played by side carbohydrate chains and the configuration of their glycosidic bonds. Some polysaccharides consisting of D-glucose residues connected by 1→4 and 1→6 α-glycosidic bonds (starches) are hydrolyzed by the amylases of the salivary and pancreatic glands of mammals. They are then absorbed in the small intestine and, together with mono- and disaccharides, make up the so-called accessible or digestible, carbohydrates.

The other part of polysaccharides (non-starch polysaccharides) is not hydrolyzed by amylases, and is not absorbed into the blood. Furthermore it is not partially or completely subjected to enzymatic degradation of the colon microflora. In addition to non-starch polysaccharides, a number of oligosaccharides (raffinose, stachyose, verbascosis), fructo-oligosaccharides, high molecular weight fructans (inulins), polyalcohols (sorbitol, xylitol, mannitol, etc.), polydextrose (a synthetic polymer of glucose), as well as resistant starch, to a greater or lesser extent, do not break down in the small intestine. Rather they are fermented by intestinal microflora and physiologically have much in common with dietary fiber. Moreover, some authors refer to dietary fibers pentosans, amino sugar of fungi and arthropods, a non-carbohydrate compound lignin and indigestible proteins. Therefore, the term “dietary fiber” includes a wider range of substances than non-digestible carbohydrates and non-starch polysaccharides. The most studied non-starch polysaccharides include pectins, alginates, carrageenans, chitosans and fucoidans. Pectins are part of the cell wall of higher plants, where they serve as a cementing material for cellulose fibers.

Many plants contain pectins in the intercellular layer between the primary cell walls, where they are involved in the regulation of the movement of water and cell juices. The primary blocks of the polymer chain of pectins are residues of D-galacturonic acid, which are connected to each other by an α (1→4) bond. The chains formed in this way number several hundred galacturon blocks. Between the galacturonic acid blocks at different distances from each other are the residues of L-rhamnose, connected to the galacturonic acid by an α (1→2) bond, as a result of which the pectin chain in this place bends by about 90°. Side chains consisting of neutral sugars, most often arabinose and galactose, originate from the main linear chain of rhamnogalacturonans (RGs). The arabinanic, galactonan and arabinogalactan side chains are connected to the rhamnose (1→4) bond.

The remains of arabinose are interconnected by a (1→5) bond, and galactose (1→4), although there are (1→3) and (1→6)-connections as well. There are D-galactopyranose L-arabinofuranose, D-xylopyranose, D-glucopyranose and L-fucopyranose but very rare. D-apiosis, 2-O-methyl-D-xylose and 2-O-methylfucose. Typically, the side chains of neutral sugars have a length of 8 to 20 molecules, and they account for 10.15% of the mass of pectin.

Pectins are distinguished by high methoxylated and low methoxylated. Pectin is considered methoxylated when the carboxyl groups of the galacturonic acid residues are esterified with methyl alcohol. The more such groups are present in the polymer chain of pectin, the higher the degree of esterification or methoxylation, and vice versa. High methoxylated pectins are characterized by a degree of esterification of more than 50% (usually from 60 to 80%), and low methoxylated, less than 50% (usually 30-40%) [6]. Alginic acid and its salts are found mainly in marine brown algae (Phaeophyta), in which they form the bulk of polysaccharides, reaching 40% dry weight, as well as in red algae of the Corallinaceae family. It is now known that bacteria belonging to the genera Pseudomonas and Azotobacter contain acetylated alginates. In algal thalli, phycocolloids are the primary components of the cell walls and extracellular matrix, playing the role of a “skeleton” and providing tissue strength and flexibility. Alginic acid consists of residues of β-D-mannuronic and α-L-guluronic acids, connected by (1→4) bonds.

The polymer thread of alginates consists of homopolymer polymannuronic and polyguluronic regions, or blocks. Between these blocks e alternating residues of both acids can be located. With polyvalent metals, alginic acid forms several types of alginates. In complete alginates, all carboxyl groups are bound to cations. Such alginates are insoluble in water. Incomplete alginates can be soluble and insoluble in water. At the same time, complete alginates of monovalent metals are highly soluble in water and form viscous, sticky solutions. Soluble salts include potassium, sodium, as well as magnesium and ammonium. Alginates can be monocationic when cations of one metal are involved in the formation of alginate, and polycationic. with cations of several metals. The source of carrageenan is red algae belonging to the families of Gigartinaceae, Solieriaceae, Rhabdoniaceae, Hypneaceae, Phyllophoraceae, Petrocelidaceae, Caulacanthaceae, Cystocloniaceae, Rhodophyllidaceae, Furcellariaceae, or Tichocarpaceae D. Carrageenans are sulfated galactans containing D-galactose and its derivatives, the remains of which are connected by regularly alternating β (1→4) and α (1→3) bonds. The 4-O-substituted carrageenan residue can be both galactose and its 3,6-anhydro derivative, and various hydroxyl groups can be sulfated.

Regular polysaccharides, the molecules of which are built from disaccharide repeating units of the same type, have their own names. Thus, several “ultimate”, or idealized, carrageenan structures have been established. This allows us to divide them into types that differ in the content of 3,6-anhydro-galactose, location and amount of sulfate groups. In accordance with the structural features of the repeating unit, 6 main types of carrageenan are distinguished: κ, λ, ι, ν, μ and θ. The carrageenans μ, ν, and λ, can be converted, respectively, into κ-, ι-, and θ-carrageenans with an alkaline or enzymatic modification. However, real natural polysaccharides rarely correspond to such idealized structures; usually a combination of two or more ultimate structures in one polymer molecule is observed. According to the modified nomenclature of red algal galactans, a code system of capital letters is used to designate a hybrid or “masked” polysaccharide structure [33]. The precursor of chitosan is the polymer N-acetyl-D-glucosamine (chitin), which is synthesized in animals, mainly crustaceans, mollusks and insects. They are also an important component of the exoskeleton, and in some fungi as the main fibrillar cell wall polymer.

Chitosan as a polymer of β-(1→4)-2-acetamido-2-deoxy-D-glucopyranose is obtained by alkaline deacetylation of chitin. Fucoidans are complex sulfated polysaccharides found in brown seaweed, in the eggs of sea urchins, and in the body wall of cucumbers. The core chain of fucoidan consists of L-fucose residues connected by α (1→3) bonds with sulfate groups in the 4th position on some fucose residues. Other fucose residues are attached to this polymer, forming branch points through the α (1→2)- or α (1→4) bonds. A small amount of other sugars, such as xylose, galactose, mannose and glucuronic acid, were also noted in fucoidan.

Known pharmaceutical composition (U.S. Pat. No. 5,773,033 “Autologous isolated and purified fibrinogen with biocompatible anionic or cationic chitosan polymer”), which is a chitosan/fibrinogen containing hemostatic agents. Agents containing fibrinogen and chitosan having strong hemostatic properties and are applicable in urgent cases to stop bleeding from damaged vessels are patented. Chitosan and fibrinogen are applied to the tissue base and, when bleeding occurs, similar tissue is applied to the wound, thereby stopping the bleeding.

The disadvantage of this invention is the presence of an expensive protein of fibrinogen originating from donated blood in a lyophilized form. In addition, chitosan and its salts have a very limited degree of swelling and the low rate onset of action. Such compositions cannot be used to urgently stop heavy gunshots, including abdominal bleeding due to the limited rate onset of action after 15-20 minutes. After this period of time with arterial bleeding, the patient loses more than 50% of their blood and dies. In addition, these compositions are not able to activate tissue regeneration.

These disadvantages are eliminated by using combinatorial mixtures of binary modified polysaccharide derivatives, including in the form of salts.

The low cost of these products, the formation of salts between them and the carboxy-polysaccharide carrier during swelling, a high swelling rate and a high percentage of liquid uptake make it possible to use this composition in the form of a powder when it introduced into the wound to stop urgent bleed, including arterial bleeding. The super-effect of the proposed derivatives of polysaccharides is almost instant by swelling (within 5 seconds it absorbs 10 volumes of liquid).

The aim of the invention is the creation of a combinatorial mixture of biologically active derivatives of polysaccharides and pharmaceutical compositions based on them. These compositions will have following effects: hemostatic, wound healing, antiviral, immunomodulatory effects, capable of exerting a quick effect in emergency cases with rupture of blood vessels, including gunshot and including with arterial bleeding with local application in the form of a sterile powder.

### DISCLOSURE OF INVENTION

This goal is achieved by creating an indivisible combinatorial mixture of a biologically active derivative of a polysaccharide (polysaccharides) and based on them pharmaceutical compositions with a hemostatic, wound healing, antiviral and immunomodulating action, containing as the main active substance an indivisible supramolecular combinatorial mixture of substituted glucopyranose polymer derivatives, obtained by simultaneous combinatorial modification of the polysaccharide by at least two covalent modifiers. As a result of the synthesis, a combinatorial mixture with the maximum number of combinations of modified polysaccharide derivatives is formed, and as a biologically active substance to obtain a pharmaceutical composition, a whole combinatorial mixture of polysaccharide derivatives is used in the form of a supramolecular structure without separation into individual components. Additional differences of the invention are that:
the molar ratio of polysaccharide and covalent modifiers in the combinatorial synthesis reaction is calculated by the formulas:

k=n×(2ⁿ−1) (1)

m=4×(3×2ⁿ⁻²−1), (2)

n=the number of groups available for substitution in the polysaccharide (in terms of monomer, a derivative of glucopyranose);
m=the number of moles of the starting polysaccharide and the number of different molecules of combinatorial derivatives after synthesis (in terms of the glucopyranose derivative);
k=the number of moles of each of the two modifiers in the combinatorial synthesis reaction to obtain the maximum number of different derivatives;
as the starting polymer for subsequent combinatorial binary synthesis, cellulose, carboxymethyl cellulose, carboxymethyl propyl cellulose, heparin, chitosan, succinyl-chitosan, carboxymethylchitosan, starch, carboxymethyl starch, methyl starch, heparin are used;
additionally contains lysine amino acid base;
pharmaceutical compositions based on the obtained combinatorial derivatives have immunomodulatory, antiviral, wound healing and hemostatic effects.

### BRIEF DESCRIPTION OF THE DRAWINGS

FIG. 1, The chemical structure of heparin (CAS 9041-08-1, Mr=1134.899 g/mol, n=6), contains 6 equivalent hydroxyl groups available for modification in the structure of glucopyranose.

### EMBODIMENTS OF THE INVENTION

Example 1. Obtaining a combinatorial mixture K1 based on starch derivatives 0.1-90 kg of starch is added to the mixer, 1-900 L of hot water is added, the solution is then stirred until the polysaccharide is completely dissolved. The solution is then cooled to room temperature, 0.02-10 kg of succinic anhydride and 0.02-10 kg of maleic anhydride are added. Then the solution is stirred until the anhydrides are completely dissolved. 1-500 L of 96% ethanol (or methanol) is added to the solution, left for a day. Lastly, the precipitate is filtered off and dried, then used as option K1 in pharmaceutical compositions.

Instead of starch, other unsubstituted or monosubstituted derivatives of starch can be used: carboxy starch, succinyl starch, maleinyl starch, carboxymethyl starch, and a mixture thereof. Also, based on the obtained combinatorial mixture of starch, salts with metals or amines can be obtained by standard methods known to an ordinary specialist in their field.

### Example 2. Obtaining a Combinatorial Mixture K2 Based on Cellulose Derivatives

In a mixer combine m mole of carboxymethyl cellulose (in terms of monomer—monocarboxymethyl-glucose), and 5 m mole of hot water and 5 m mole of ethanol. The mix solution until the polysaccharide is completely dissolved. After that, the solution is cooled to room temperature. K mol of succinic anhydride and k mol of methyl chloride are then poured in to the solution. Next the solution is stirred until the modifiers are completely dissolved. 5 m mol of ethanol 96% ethanol (or methanol) is added to the solution and left for a day. Then the precipitate is filtered off and dried, then used as option K2 in pharmaceutical compositions.

The calculation of the molar ratio of modifiers and polysaccharide is carried out according to the formulas:

k=n×(2ⁿ−1) (1)

m=4×(3×2ⁿ⁻²−1), (2)

n=the number of groups available for substitution in the polysaccharide (in terms of monomer, a derivative of glucopyranose, n=4 for glucopyranose, average chain length of 10 units, respectively, for CMC n=40);
m=number of moles of the starting polysaccharide and the number of different molecules of combinatorial derivatives after synthesis (in terms of the glucopyranose derivative) m=3298534883324 (3.3*10¹²)
k=the number of moles of each of the two modifiers in the combinatorial synthesis reaction to obtain the maximum number of different derivatives (for CMC=43980465111000 or 4.4*10¹³ mol of each modifier)

This means that to obtain a combinatorial mixture with a maximum number of different derivatives, which will be 3298534883324 molecules (or mol) to the mixture, you need to take 3298534883324 (or 3.3*10¹²) moles of polysaccharide (with an average number of chain units=10 with 4 available hydroxyl groups in each) and 43980465111000 mol of each of the modifier (4.4*10¹³ mol). Thus, the molar ratio polysaccharide: modifier No. 1: modifier No. 2 is 1:13:13. In this case, a combinatorial mixture of 3.3*10¹² different molecules of polysaccharide derivatives with different positions of substituents and different degrees of substitution of molecules is formed. Such a mixture cannot physically be divided into separate components, and in aqueous solutions forms a complex supramolecular structure through hydrogen and ionic bonds. The biological activity of the derivatives is due precisely to the supramolecular structure, and not to the individual component.

This structure of many similar, but different polysaccharides resembles a mixture of immunoglobulins and glycoprotein adhesins with immunomodulatory effects. Existing methods of physicochemical analysis are not able to identify 3.3*10¹² different molecules in one mixture. A distinctive feature of this structure mainly the presence of unusual biological (pharmacological) properties, in contrast to the starting polysaccharides.

The application of classical physicochemical methods which utilize the determination of the monomer sequence, substitution sites are not relevant, because the structure of the polysaccharide was originally known and proved by us, and it makes no sense to determine the place of substitution, because the substitution is carried out according to the principle of combinatorics in a random order, and the derivatives are distributed according to quantity based on the normal distribution. The medians of the normal distribution can be shifted to the right or left depending on the degree of accessibility of a particular group, but physicochemical analyzes of such structures have not yet been developed.

When the molar ratio of polysaccharide modifiers is changed in the direction of increasing the number of modifiers, by more than 13 mol per 1 mol of polysaccharide, completely substituted derivatives are synthesized in place of different combinatorial derivatives in a much smaller amount. A similar pattern is observed with a decrease in the number of modifiers of less than 13 mol per 1 mol of polysaccharide. In this case, a significant decrease in the number of various derivatives due to the presence of unsubstituted derivatives is also observed. The maximum activity is possessed not by individual derivatives, but by the supramolecular structure of them. This structure is stabilized only at the peak of the synthesis of the maximum variety of derivatives, that is, with a polysaccharide: modifier ratio of 1:13:13.

Instead of carboxymethyl cellulose, other unsubstituted or monosubstituted cellulose derivatives may be used: carboxy starch, succinyl starch, maleinyl starch, carboxymethyl starch, or a mixture thereof: succinyl cellulose, maleinyl cellulose, carboxymethyl cellulose, propyl cellulose, and cellulose cellulose.

Also, based on the obtained combinatorial mixture of cellulose, salts with metals or amines can be obtained by standard methods known to an ordinary specialist in their field.

### Example 3. Obtaining a Combinatorial Mixture of K3 Based on Heparin Derivatives

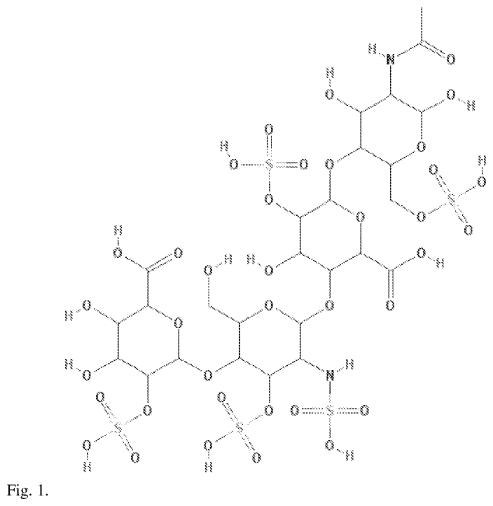

In a mixer combine m mol (CAS 9041-08-1, Mr=1134.899 g/mol, n=6) of heparin (FIG. 1), 5 m mol of hot water and 5 m mol of ethanol. Then mix the solution until the polysaccharide is completely dissolved and the solution is cooled to room temperature. After that pour k mol of succinic anhydride and k mol of maleic anhydride into the solution. The solution is then stirred until the modifiers are completely dissolved. 5 m mol of ethanol 96% ethanol (or methanol) is then added to the solution. It is then left for a day, the precipitate is filtered off and dried, then used as option K2 in pharmaceutical compositions.

The calculation of the molar ratio of modifiers and polysaccharide is carried out according to the formulas:

k=n×(2ⁿ−1) (1)

m=4×(3×2ⁿ⁻²−1), (2)

n=the number of groups available for substitution in the polysaccharide (for a given heparin polysaccharide n=6);
m=number of moles of the starting polysaccharide and the number of different molecules of combinatorial derivatives after synthesis (for heparin) m=188
k=the number of moles of each of the two modifiers in the combinatorial synthesis reaction to obtain the maximum number of different derivatives (for heparin=378 mol of each modifier)

This means that to obtain a combinatorial mixture with a maximum number of different derivatives, which will be 188 heparin derivatives, you need to take 188 moles of heparin (with 6 groups available for modification) and 378 mol of each of the modifier. Thus, the molar ratio polysaccharide: modifier No. 1: modifier No. 2 is 1: 2: 2. In this case, a combinatorial mixture of 188 different molecules of heparin derivatives with different positions of substituents and different degrees of substitution of molecules is formed. Such a mixture in aqueous solutions forms a complex supramolecular structure through hydrogen and ionic bonds. The biological activity of the derivatives is due precisely to the supramolecular structure, and not to the individual component. This structure of many similar, but different polysaccharides resembles a mixture of immunoglobulins and glycoprotein adhesins with immunomodulatory effects. Subsequently, samples K1, K2, K3 were studied for several types of biological activity. Existing methods of physicochemical analysis are not able to identify many different molecules in one mixture, if the molecules are similar in molecular weight. A distinctive feature of this structure is only the presence of unusual biological (pharmacological) properties, in contrast to the starting polysaccharides. Also, based on the obtained combinatorial mixture of heparin, salts with metals or amines can be obtained by standard methods known to an ordinary specialist in their field.

Instead of heparin, such natural polysaccharides can be used, like: inulin, pectins, gums, mucus, alginic acid, and chitosan.

NMR C₁₃: CH: s: 106.1; 105.8; 104.3; 95.4; 78.5; 77.0; 75.6; 79.5; 78.8; 86.8; 80.1; 77.5; 78.2; 71.8; 73.8; 69.2; 66.1; 58.1; 56.1; C: m 166.5-174.7; CH2: 62.2; 68.2; 29.5; 29.1; 23.6; CH: 134.9; 136.1

Based on their NMR spectrum, we can confidently say that there are residues of succinic and maleic acids in the combinatorial mixture, and the formation of a complex supramolecular structure is confirmed by the presence of a continuous multiplet bands in the range 166.5-174.7. A similar picture is characteristic of complex supramolecular structures of catenanes and rotaxanes.

To test the antiviral activity of the synthesized heparin derivatives with different ratios of components in the combinatorial synthesis reaction, the antiviral activity of the derivatives was studied. They were studied by the screening method on models of the H1N1 (Inf) influenza virus. reference strain of vesicular stomatitis virus (Vesic.—VVS) and herpes simplex virus type 1 (Herp.—strain L-2) in tablets on chicken fibroblast culture according to the degree of degradation (cytopathic effect, detachment from the bottom of the hole). The degree of “desquamation” of the cells was determined by staining the culture with a vital dye, the concentration of which was determined spectrophotometrically with respect to a healthy monolayer and an empty well. The results of in vitro studies are shown in table 1.

TABLE 1
Antiviral activity of supramolecular combinatorial
derivatives of heparin K3 obtained in the reaction
with different molar ratio of modifiers

| | | | | % cytoprotective antiviral | | |
|---|---|---|---|---|---|---|
| | The molar ratio of reagents * | | | activity ** | | |
| No p/p | m | k1 | k2 | Inf | Herp | Vesic |
| 1 | 188 | 1512*** | 1512*** | 0 | 0 | 0 |
| 2 | -//- | 756 | 756 | 50 | 45 | 45 |
| 3 | -//- | 378 | 378 | 100 | 90 | 100 |
| 4 | -//- | 94 | 94 | 59 | 30 | 45 |
| 5 | -//- | 47 | 47 | 0 | 0 | 0 |
| 6 | -//- | 23 | 23 | 0 | 0 | 0 |
| 7 | -//- | 12 | 12 | 0 | 0 | 0 |
| 8 | -//- | 6 | 6 | 0 | 0 | 0 |
| 9 | -//- | 3 | 3 | 0 | 0 | 0 |
| 10 | -//- | 1 | 1 | 0 | 0 | 0 |
| 11 | -//- | 0 | 0 | 0 | 0 | 0 |
| 12 | -//- | 1512*** | 0 | 0 | 0 | 0 |
| 13 | -//- | 756 | 0 | 0 | 0 | 0 |
| 14 | -//- | 378 | 0 | 0 | 0 | 0 |
| 16 | -//- | 94 | 0 | 0 | 0 | 0 |
| 17 | -//- | 47 | 0 | 0 | 0 | 0 |
| 18 | -//- | 23 | 0 | 0 | 0 | 0 |
| 19 | -//- | 12 | 0 | 0 | 0 | 0 |
| 20 | -//- | 6 | 0 | 0 | 0 | 0 |
| 21 | -//- | 3 | 0 | 0 | 0 | 0 |
| 22 | -//- | 1 | 0 | 0 | 0 | 0 |
| 23 | -//- | 0 | 1512*** | 0 | 0 | 0 |
| 24 | -//- | 0 | 756 | 0 | 0 | 0 |
| 25 | -//- | 0 | 378 | 0 | 0 | 0 |
| 26 | -//- | 0 | 94 | 0 | 0 | 0 |
| 27 | -//- | 0 | 47 | 0 | 0 | 0 |
| 28 | -//- | 0 | 23 | 0 | 0 | 0 |
| 29 | -//- | 0 | 12 | 0 | 0 | 0 |
| 30 | -//- | 0 | 6 | 0 | 0 | 0 |
| 31 | -//- | 0 | 3 | 0 | 0 | 0 |
| 32 | -//- | 0 | 1 | 0 | 0 | 0 |
| 33 | -//- | 3024*** | 0 | 0 | 0 | 0 |
| 34 | -//- | 1512 | 1 | 0 | 0 | 0 |
| 35 | -//- | 756 | 3 | 0 | 0 | 0 |
| 36 | -//- | 378 | 6 | 0 | 0 | 0 |
| 37 | -//- | 94 | 12 | 0 | 0 | 0 |
| 38 | -//- | 47 | 23 | 40 | 35 | 30 |
| 39 | -//- | 23 | 47 | 0 | 0 | 0 |
| 40 | -//- | 12 | 94 | 0 | 0 | 0 |
| 41 | -//- | 6 | 378 | 0 | 0 | 0 |
| 42 | -//- | 3 | 756 | 0 | 0 | 0 |
| 43 | -//- | 1 | 1512 | 0 | 0 | 0 |
| 44 | -//- | 0 | 3024*** | 0 | 0 | 0 |

* m is the number of moles of heparin in the combinatorial synthesis reaction; K1 is the number of moles of succinic anhydride in the reaction; K2 is the number of moles of maleic anhydride in the reaction;
** % of the remaining monolayer of cells after infection with viruses and replacing the culture with the studied drug in the culture after 48 hours of incubation in the presence of the test substance added in a pre-selected concentration (ED90 = 0.075 μg/ml);
***the maximum molar ratio at which all groups in the polysaccharide are replaced, an excess of this ratio leads to the fact that unreacted modifiers remain in the reaction medium - succinic anhydride and maleic anhydride.

As can be seen from table 1, only with the calculated ratio of the components, when the maximum number of different heparin derivatives is formed, a biologically active and effective supramolecular structure (derivative 3 or K3) is formed, capable of completely protecting the cell monolayer (ED100) from a degrading dose of 0.075 μg/ml cytopathic action of viruses.

### Example 4. Obtaining a Pharmaceutical Composition “K1K”

In a mixer combine 0.1-90 kg combinatorial mixture of modified starch (or its salts) and 0.1-30 kg of the base of the amino acid L-lysine. Then mix until completely homogeneous. It is then packed in 1-30 g in aluminum bags or glass bottles. The bottles are corked with rubber stoppers and rolled with aluminum caps, and the aluminum bags are sealed on a packaging machine. Vials and bags are sterilized in an autoclave under standard sterilization conditions (120 0 C, 30 min).

Salts of combinatorial derivatives are prepared by known methods, which typically involve mixing K1 with either a pharmaceutically acceptable acid to form an acid addition salt or a pharmaceutically acceptable base to form a base addition salt. Whether the acid or base is pharmaceutically acceptable can be easily decided by a person skilled in the art, after taking into account the specific intended use of the compound. For example, not all acids and bases that are acceptable for ex vivo applications can be used for pharmaceutical compositions. Similarly not all acids and bases that are suitable for local use can be used parenterally.

Depending on the intended use, pharmaceutically acceptable acids include organic and inorganic acids such as formic acid, acetic acid, propionic acid, lactic acid, glycolic acid, oxalic acid, pyruvic acid, succinic acid, maleic acid, malonic acid, brown acid, sulfuric acid, hydrochloric acid, hydrobromic acid, nitric acid, perchloric acid, phosphoric acid and thiocyanic acid, which form ammonium salts with free amino groups of peptides and conjugates. Especially preferred is palmitic acid for the production of K1 salts of the invention. Pharmaceutically acceptable bases which form carboxylate salts with free K1 carboxyl groups and functional equivalents include ethylamine, methylamine, dimethylamine, triethylamine, isopropylamine, diisopropylamine and other mono, di and trialkylamines, as well as arylamines. In addition, pharmaceutically acceptable solvates are also included.

Pharmaceutically acceptable salts can be used in the invention. For example, salts of inorganic acids such as hydrochlorides, hydrobromides, phosphates, sulfates and the like; and salts of organic acids such as acetates, propionates, malonates, benzoates and the like. A full discussion of pharmaceutically acceptable excipients is available in Remington's Pharmaceutical Sciences (Mack Pub. Co., N. J. 1991). Pharmaceutically acceptable carriers in pharmaceutical compositions may contain liquids such as water, saline, glycerol, and ethanol.

In addition, auxiliary substances, such as a humectant or emulsifying agents, pH-creating substances and the like, may be present in such drug media. Parenteral pharmaceutical compositions are generally prepared as injections, or as liquid solutions or suspensions. Solid forms suitable for dissolving or forming a suspension in liquid drug media can also be prepared prior to injection. Liposomes are included in the definition of a pharmaceutically acceptable carrier. For therapeutic effects, K1 can be obtained as described above and applied to an object that needs it. K1 can be introduced into the subject by any suitable method, preferably in the form of a pharmaceutical composition adapted to such a method and in a dosage that is effective for the intended treatment.

### Example 5. Obtaining a Pharmaceutical Composition “K2K”

In a mixer combine 0.1-90 kg combinatorial mixture of modified cellulose (or its salts), and 0.1-30 kg of the base of the amino acid L-lysine. Then mix until completely homogeneous, pack 1-30 g in aluminum bags or glass bottles. The bottles are corked with rubber stoppers and rolled with aluminum caps, and the aluminum bags are sealed on a packaging machine. Vials and bags are sterilized in an autoclave under standard sterilization conditions (120 0 C, 30 min).

Salts of combinatorial derivatives are prepared by known methods, which typically involve mixing K2 with either a pharmaceutically acceptable acid to form an acid addition salt or a pharmaceutically acceptable base to form a base addition salt. Whether the acid or base is pharmaceutically acceptable can be easily decided by a person skilled in the art, after taking into account the specific intended use of the compound. For example, not all acids and bases that are acceptable for ex vivo applications can be used for pharmaceutical compositions, and not all acids and bases that are suitable for local use can be used parenterally.

Depending on the intended use, pharmaceutically acceptable acids include organic and inorganic acids such as formic acid, acetic acid, propionic acid, lactic acid, glycolic acid, oxalic acid, pyruvic acid, succinic acid, maleic acid, malonic acid, brown acid, sulfuric acid, hydrochloric acid, hydrobromic acid, nitric acid, perchloric acid, phosphoric acid and thiocyanic acid, which form ammonium salts with free amino groups of peptides and conjugates. Especially preferred is palmitic acid for the production of the K2 salts of the invention. Pharmaceutically acceptable bases which form carboxylate salts with free K2 carboxyl groups and functional equivalents include ethylamine, methylamine, dimethylamine, triethylamine, isopropylamine, diisopropylamine and other mono, di and trialkylamines, as well as arylamines. In addition, pharmaceutically acceptable solvates are also included.

Pharmaceutically acceptable salts can be used in the invention, for example, salts of inorganic acids such as hydrochlorides, hydrobromides, phosphates, sulfates and the like; and salts of organic acids such as acetates, propionates, malonates, benzoates and the like. A full discussion of pharmaceutically acceptable excipients is available in Remington's Pharmaceutical Sciences (Mack Pub. Co., N. J. 1991). Pharmaceutically acceptable carriers in pharmaceutical compositions may contain liquids such as water, saline, glycerol, and ethanol. In addition, adjuvants, such as a humectant or emulsifying agents, pH-generating substances and the like, may be present in such drug media.

Parenteral pharmaceutical compositions are generally prepared as injections, or as liquid solutions or suspensions. Solid forms suitable for dissolving or forming a suspension in liquid drug media can also be prepared prior to injection. Liposomes are included in the definition of a pharmaceutically acceptable carrier. For therapeutic effects, K2 can be obtained as described above and applied to an object that needs it. K2 can be introduced into the subject by any suitable method, preferably in the form of a pharmaceutical composition adapted to such a method and in a dosage that is effective for the intended treatment.

### Example 6. Obtaining the Pharmaceutical Composition “K3K”

In a mixer combine 0.1-90 kg combinatorial mixture of modified heparin (or its salts), and 0.1-30 kg of the base of the amino acid L-lysine. Then mix until completely homogeneous, packaged in 0.05-0.1 g in glass bottles. Bottles are corked with rubber stoppers and rolled with aluminum caps. Vials are sterilized in an autoclave under standard sterilization conditions (120° C., 30 min). You can also make a sterile 0.1-5% solution in distilled water or in a 0.9% saline, put in ampoules or syringes and sterilize by autoclaving (120 0 C, 30 min).

Salts of combinatorial derivatives are prepared by known methods, which typically involve mixing K3 with either a pharmaceutically acceptable acid to form an acid addition salt or a pharmaceutically acceptable base to form a base addition salt. Whether the acid or base is pharmaceutically acceptable can be easily decided by a person skilled in the art, after taking into account the specific intended use of the compound.

For example, not all acids and bases that are acceptable for ex vivo applications can be used for pharmaceutical compositions, and not all acids and bases that are suitable for local use can be used parenterally. Depending on the intended use, pharmaceutically acceptable acids include organic and inorganic acids such as formic acid, acetic acid, propionic acid, lactic acid, glycolic acid, oxalic acid, pyruvic acid, succinic acid, maleic acid, malonic acid, brown acid, sulfuric acid, hydrochloric acid, hydrobromic acid, nitric acid, perchloric acid, phosphoric acid and thiocyanic acid, which form ammonium salts with free amino groups of peptides and conjugates.

Especially preferred is therefore palmitic acid for the production of K3 salts of the invention. Pharmaceutically acceptable bases which form salts of carboxylates with free K3 carboxyl groups and functional equivalents include ethylamine, methylamine, dimethylamine, triethylamine, isopropylamine, diisopropylamine and other mono, di and trialkylamines, as well as arylamines. In addition, pharmaceutically acceptable solvates are also included.

Pharmaceutically acceptable salts can be used in the invention, for example, salts of inorganic acids such as hydrochlorides, hydrobromides, phosphates, sulfates and the like. Additionally, other salts are salts of organic acids such as acetates, propionates, malonates, benzoates and the like. A full discussion of pharmaceutically acceptable excipients is available in Remington's Pharmaceutical Sciences (Mack Pub. Co., N. J. 1991). Pharmaceutically acceptable carriers in pharmaceutical compositions may contain liquids such as water, saline, glycerol, and ethanol. In addition, adjuvants, such as a humectant or emulsifying agents, pH creating substances and the like, may be present in such drug media. Typically, parenteral pharmaceutical compositions are prepared as injections, or as liquid solutions or suspensions; solid forms suitable for dissolution or suspension in liquid drug media can also be prepared prior to injection. Liposomes are included in the definition of a pharmaceutically acceptable carrier.

K3 can be introduced into the object by any suitable method, preferably in the form of a pharmaceutical composition adapted to such a method and in a dosage that is effective for the intended treatment.

### Example 7. The Effect of the Drug “K1K” and “K2K” on the Time of Blood Coagulation

The most difficult type of surgical pathology are wound injuries of the abdomen, accompanied by heavy bleeding. In this regard, the provision of reliable hemostasis is one of the most pressing problems of modern surgery.

As a result of the studies, the bleeding time was established under the conditions of using modern hemostops. The following application preparations were used as materials for experimental studies: hemostatic collagen sponge (HHC), “Hemostop”, “Celox”, and experimental preparations “K1K” and “K2K”. The experiment was performed on 60 Wistar male rats. In an acute experiment, a median laparotomy was performed under anesthesia, and standard liver and spleen injuries were modeled.

A hemostatic agent was poured onto the wound area and amount of it based on wound size. Simultaneously with the modeling of wounds using a stopwatch, the bleeding time began. Thus, it was found that all experimental materials have hemostatic activity, significantly shortening bleeding time, with the exception of the experiment with the Hemostop material, the indicators of which are approaching control. The bleeding time from liver injury in the conditions of application of materials “K1K” and “K2K” decreased by 89.5-93.5% relative to the control and by 42.1-44.9% relative to the HHC.

The shortening of the time to stop bleeding based on a standard spleen injuries was maximum when testing the materials “K1K”, “K2K” and was 3.43-3.55 times (p<0.001) less than the control and 2.80-2.89 times (p<0.05)—relative to HHC. Indicators of bleeding time for GGK material contributed to a decrease in the time for stopping bleeding from the liver injury by 37.0-42.4% and from spleen injury by 22.3-27.4% relatively compare to the control.

### Example 8. The Resorption Rate “K1K” and “K2K”

One of the most important indicators of hemostatic drugs is their biological inertness and complete controlled biodegradation. Accordingly, as a result of in vitro studies, the rate of resorption of modern hemostatic materials K1K and K2K was established. Materials for the study were hemostatic application products: hemostatic collagen sponge (HCS), Cellox, Hemostop, as well as new materials on K1K and K2K. The study was carried out in vitro under experimental conditions. A sample of hemostatic material weighing 1 g was placed in a volumetric tube containing 5 ml of distilled water. The tube was placed in a thermostat with a constant temperature of 37° C. The results of the biological degradation rate of each hemostatic material were evaluated on days 1, 3, 7, and 14. The test hemostatic tube was removed from the thermostat and a visual descriptive evaluation of the hemostatic agent was performed. The studied hemostop was removed from the experimental medium and dried. Subsequently, repeated weighing of the studied hemostop was performed. The difference in the mass of the hemostop before the experimental study and after its implementation, expressed as a percentage, reflected the rate of resorption of the studied drug. Studying in an experiment in vitro the rate of degradation of hemostatic agents showed that all the studied samples of materials were resorbed. High resorptive activity was observed in the K2K hemostatic composition—100% (P≤0.001), resorption rate—98.73% (P≤0.001), the maximum resorptive activity was observed in the HCS and K1K preparations. The lowest rates of degradation were noted in the study of the Hemostop material, the resorption of which is 10 times less relative to the Celox materials (42.3% (P≤0.05)) amounted to 10.34% (P≤0.05).

### Example 9. The Study of the Sorption Activity of “K1K” and “K2K”

As a result of the study, the sorption properties of the modern application hemostops “K1K” and “K2K” were evaluated. The following hemostatic samples were examined: hemostatic collagen sponge, “Celox”, “Hemostop”, “K1K” and “K2K”. During the experiment, the mass of distilled water was determined, which is capable of absorbing a prototype of the studied materials of standard equal mass (1 g). The degree of complete saturation of the studied agent was determined visually by a change in the spatial properties of the material—swelling. The time of complete saturation of the application preparations was fixed using a stopwatch. To assess the sorption activity of the studied samples of materials, their hygroscopicity was determined using the following formula: hygroscopicity (ml/g)=m1/m2, where: m1 is the volume (mass) of water absorbed by the material (ml); m2 is the mass (g) of material. For a comprehensive assessment of the sorption properties of application materials, we used a sorption indicator (SP), which is the volume of liquid that 1 g of a material sample can absorb for 1 s: SP (ml×s/g)=hygroscopicity/t, where: t—time of complete saturation of the material (s). The obtained data were processed statistically with the calculation of average values, average errors of the average and significance of differences using the Student and Mann-Whitney criteria (with respect to the hemostatic collagen sponge). The error of the statistical hypothesis was p≤0.05. Thus, a relatively high sorption activity was demonstrated by a hemostatic collagen sponge having hygroscopicity of 69.41±1.65 ml/g and a sorption index of 15.1±0.95 ml×s/g. The hygroscopicity of the K1K and K2K materials was 78.62±2.18 ml/g (p≤0.05) and 88.3±2.11 ml/g (p≤0.05), and the sorption the indicator is 23.8±1.24 ml×s/g (p≤0.05) and 25.5±1.41 ml×s/g (p≤0.05), respectively. The minimum sorption properties were noted in the hemostops “Celox” and “Hemostop”, the hygroscopicity of which amounted to 5.63±1.21 ml/g and 6.11±1.16 ml/g, and the sorption index was 1.23±0.11 ml×s/g and 1.10±0.04 ml×s/g, respectively.

### Example 10. Determination of the Effect of the Compositions “K1K” and “K2K” on Tissue Regeneration

The study of the healing properties of the compositions was carried out on male Vistar white rats. In 38 animals that were previously anesthetized, on the dorsal side of the body, behind the right shoulder blade, a skin area of 2 by 2 cm was cut. The skin was taken with tweezers and pulled, a skin fragment of 2 cm was cut, the depth of the wound was 2 mm, the average area of the wound was 4±1.0 cm2. The resulting wounds of a polygonal shape were intensively bleeding. Then the animals of the first and second groups (10 in each) were applied “K1K” and “K2K” to the wound. The wounds of rats of the 3rd group were treated with “Celox” The 4th group of 8 animals was the control group, the wounds of these animals were not treated. The preparations were applied in such a way that the formed gels covered the entire surface of the wound and capture a small fragment around the wound. BF-6 glue was applied on top of the gel, and. The animals were then released into cells. After 3, 6, 9, 11, and 13 days from the start of the experiment (before the healing of wounds in animals of all groups), a planimetric study was carried out, which made it possible to judge the features of the reparative processes. The measurement of the area of the wounds was carried out in this way: its contours were applied to the celluloid film that was applied to the wound, after which the area of the wound surface was determined using graph paper. The results of the first series of experiments (Table 2) showed that wound healing at all stages of the study was significantly accelerated under the influence of the K1K and K2K compositions. The effectiveness of the K2K composition was statistically higher than that of the K1K and Celox compositions.

TABLE 2
Rat wound healing in rats under the influence of compositions K1K and K2K

| | | | Wound Area * (S) | | | | |
|---|---|---|---|---|---|---|---|
| | | | during the observation, cm2 (M ± m) | | | | |
| | | | 1-3 | 3-6 | 6-9 | 9-11 | 11-13 |
| Substance | The basis | n | days | days | days | days | days |
| K2K | Combinatorial binary | 10 | 4.2 ± 0.6 | 1.2 ± 0.2 | 0.2 ± 0.1 | — | — |
| | cellulose derivative | | | | | | |
| K1K | Combinatorial binary | 10 | 4.2 ± 0.6 | 1.8 ± 0.2 | 0.6 ± 0.2 | 0.4 ± 0.1 | — |
| | starch derivative | | | | | | |
| Celox | Chitosan | 10 | 4.0 ± 1.1 | 3.5 ± 0.3 | 2.6 ± 0.4 | 1.2 ± 0.3 | 0.3 ± 0.1 |
| Control | — | 8 | 4.0 ± 0.6 | 3.6 ± 0.6 | 2.6 ± 0.6 | 1.5 ± 0.5 | 0.5 ± 0.2 |

* P [symbol] 0.05 As can be seen from table 2, the wounds in animals were almost 2 times faster to heal, the wounds of which were treated with K2K composition (from 13 to 6 days), while the efficiency of the control sample Celox did not differ from the control. Wound epithelization was initiated already on the second day after application of the composition.
Example 11. The study of the antiviral effect of drugs K1K, K2K and K3K on influenza A virus (N3 N2)

Aqueous K2K solutions in various doses (ten-fold dilutions) were administered to 15 chicken embryos in the allantoic cavity in a volume of 0.2 ml 12 hours after the virus was introduced in a working dose (100 TCE_{50/0.2 ml}). Each experiment was accompanied by control of the test virus in the working dose. Infected and non-infected (control) embryos were incubated at 360° C. for 48 hours. Then, the embryos were opened, from which the allantoic fluid was aspirated. Titration of the virus in allantoic fluid was carried out according to the generally accepted method with 1% red blood cells of 0 (1) human blood group. Defined coefficient of protection (KZ). The virus titer in the experimental and control groups of chicken embryos is presented in tables 3-5.

TABLE 3
The effective concentration of K1K in the
model of influenza infection in vivo.

| | The | | | Minimum |
|---|---|---|---|---|
| | concentration | Virus titer | | effective |
| | of the drug | (lg TCE_{50/ml}) | | concentration |
| Group | (mg/mL) | experimental | Control | (MEC mg/mL) |
| Control (0.9% | — | 12 | 12 | — |
| sodium | | | | |
| chloride | | | | |
| solution | | | | |
| was injected) | | | | |
| Experimental | 50 ± 5 | 0 | 12 | 0.5 |
| | 5 ± 1 | 2 | 12 | 5 |
| | 0.5 ± 0.05 | 4 | 12 | |
| | 0.05 ± 0.005 | 8 | 12 | |
| | 0.005 ± 0.0005 | 10 | 12 | |

TABLE 4
Effective K2K concentration in the
in ovo influenza infection model

| | The | | | Minimum |
|---|---|---|---|---|
| | concentration | Virus titer | | effective |
| | of the drug | (lg TCE_{50/ml}) | | concentration |
| Group | (mg/mL) | experimental | Control | (MEC mg/mL) |
| Control (0.9% | — | 12 | 12 | — |
| sodium | | | | |
| chloride | | | | |
| solution | | | | |
| was injected) | | | | |
| Experimental | 50 ± 5 | 0 | 12 | 0.5 |
| | 5 ± 1 | 2 | 12 | 5 |
| | 0.5 ± 0.05 | 4 | 12 | |
| | 0.05 ± 0.005 | 8 | 12 | |
| | 0.005 ± 0.0005 | 8 | 12 | |

TABLE 5
The effective concentration of K3K in
the model of influenza infection in ovo

| | The | | | Minimum |
|---|---|---|---|---|
| | concentration | Virus titer | | effective |
| | of the drug | (lg TCE_{50/ml}) | | concentration |
| Group | (mg/mL) | experimental | Control | (MEC mg/mL |
| Control (0.9% | — | 12 | 12 | — |
| sodium | | | | |
| chloride | | | | |
| solution | | | | |
| was injected) | | | | |
| Experimental | 50 ± 5 | 0 | 12 | 0.05 |
| | 5 ± 1 | 0 | 12 | 5 |
| | 0.5 ± 0.05 | 1 | 12 | |
| | 0.05 ± 0.005 | 3 | 12 | |
| | 0.005 ± 0.0005 | 6 | 12 | |

As can be seen from tables 3-5, the K3K composition based on heparin turned out to be the most effective. The minimum effective concentration of K3K against the influenza virus, which completely inhibits the synthesis of the virus, is 50 ug/mL. With an increase in dilution of the drug, the effectiveness of K3K decreases and has a dose-dependent character. This fact indicates the presence of a direct antiviral effect in the K3K preparation with respect to the H3N2 influenza virus. Other combinatorial derivatives also had antiviral activity, but at higher doses.

### Example 12. The Study of the Antiviral Effect of the Compositions K1K, K2K, K3K on Cytopathic Viruses (Vesicular Stomatitis Virus, Coronavirus, Herpes Simplex Virus Type 1)

Antiviral activity against this group of viruses was determined in a culture of the above cells. The reaction was carried out in the following way: 0.2 ml of the corresponding virus in a working dose (100 TCE_{50/0.2 mL}) was added in a volume of 0.2 ml in a 2-day washed cell culture. 0.8 mL of support medium was added. When the CPP appeared in the culture, drugs were introduced in various doses. As a control, the same was done with test viruses without the drug. Cells were incubated at 37° C. in an incubator. The experience was recorded on 3.5.7 days. The decrease in virus titer under the influence of the test drug by 21 g or more in comparison with the control was evaluated as a manifestation of antiviral activity. The results of the study of antiviral activity of the drugs are presented in table 6

TABLE 6
The study of the antiviral effect of the drug KR against viruses:
vesicular stomatitis, coronavirus, herpes simplex virus type 1)

| | | | The maximum drop in the titer of |
|---|---|---|---|
| Substances | Virus | MEC, ug/mL | the virus, lg TCE_{50/mL} |
| K1K | VVS | 5000 | 4.3 |
| | CV | 5000 | 3.9 |
| | HSV1 | 5000 | 4.9 |
| K2K | VVS | 500 | 4.4 |
| | CV | 500 | 3.8 |
| | HSV1 | 500 | 4.8 |
| K3K | VVS | 50 | 4.6 |
| | CV | 50 | 4.4 |
| | HSV1 | 50 | 4.6 |

As can be seen from table 6, all combinatorial derivatives of polysaccharides have antiviral activity and the ability to suppress the reproduction of all the viruses we studied in concentrations from 50 to 5000 ug/mL 0.05 mg/mL. The most interesting for introduction is the composition K3K, whose CTI is 1000. In addition, all the compositions were active against all the viruses studied, while not one drug of comparison showed such activity. Thus, the drug is not associated with specific characteristics of the virus or cell culture, but affects the mechanisms common to all cells.

### Example 13. The Study of the Antiviral Effect of Pharmaceutical Compositions K1K, K2K, K3K In Vitro on Models of Viruses of Farm Animals

The tests were performed in 96-well panels with porcine transmissible gastroenteritis virus (TGV) strain D-52 with an initial titer of 104.0 TCD50/mL (tissue cytopathic doses) in a transplanted piglet testicle cell culture (PTP) and large diarrhea virus cattle strain “Oregon” with an initial titer of 10⁷ TCE_{50/mL} in transplanted culture of saiga kidney cells (PS). When testing the viral-static (inhibitory) action, cell cultures were infected with viruses at doses of 100 and 10 TCE U/ml and incubated in an incubator at 37° C. KR2 was then introduced into the cell cultures (CC) at various doses 1-1.5 hours after infection (after adsorption period). For each dilution took 8 well panels.

After making the compound, the cell cultures were incubated at 37° C. for 72-144 hours until a clear manifestation of CPE (cytopathogenic effect) was observed in the control of viruses. Controls were cell cultures infected with the virus, inactive KK and KK, where only various concentrations of experimental compositions were added. Virusstatic effect was determined by the difference in titer of viruses in the experiment and control. When determining the virucidal (inactivating) effect, different doses of the compositions were mixed in equal volumes with the virus-containing material and incubated in an incubator at 37° C. for 24 hours. A virus-containing material was used as a control, to which a placebo (0.9% sodium chloride solution) and intact cell cultures were added instead of a compound solution. The mixture after contact was titrated in parallel with the control.

The results were measured at 72-144 hours after incubation at 37° C., after a clear manifestation of CPE in virus controls. The virucidal effect was determined by the difference in virus titers in the experiment and control and expressed in lg TCD50. As a result of the studies, it was found that the K3K composition at a concentration of 50 μg/ml suppressed the reproduction of the TGV virus by 2.90 lg TCE 50/ml, at an infectious dose of 100 TCD50/ml and in the same dose by 4.15 lg TCE U/ml, an infectious dose of 10 TCD50/ml. At a dose of 50 μg/ml, K3K inactivated the TGS virus on 4.0 lg TCE 50/ml. Composition K3K at a dose of 50 μg/ml inactivated cattle diarrhea virus by 4.41 g TCE 50/ML.

Therefore, the K3R compound has the most pronounced virostatic (inhibitory) and virucidal (inactivating) effects on TGV viruses and cattle diarrhea; on this basis, it is possible to create chemotherapeutic agents for the treatment and prevention of infectious diseases of viral etiology. Derivatives from the compositions K1K and K2K had weak activity and showed it only in doses of 500-5000 μg/ml

### Example 14. The Study of the Antiviral Activity of K3K in an Animal Experiment (Herpes Virus Kerato-Conjunctivitis/Encephalitis in Rabbits)

The features of the experimental system and its level of adequacy to a natural human disease undoubtedly play a decisive role in assessing the effect of antiviral substances on the course of infection. Herpetic experimental infection is of interest due to the fact that herpetic diseases are widespread and extremely variable in clinical manifestations. Models of experimental herpes in animals are finding wider application in the study of new antiviral substances. As you know, one of the clinical forms of systemic herpes is herpetic encephalitis, which is reproduced in guinea pigs, hamsters, rats, mice, rabbits, dogs, monkeys. Herpetic keratoconjunctivitis in rabbits with an average weight of 3.5 kg was obtained by applying infectious material (herpes simplex virus type 1 strain L-2) on a scarified cornea. The animal was restraint, and eye anesthesia was performed with lidocaine (instilled into the eye). Eyelids were opened, and several scratches were applied to the cornea using a syringe needle. Then the virus-containing material was introduced and, closing the eyelids, rubbed it into the cornea in circular motions. Dose of the virus: 0.05 ml. 16 rabbits were used in the experiment, ten of them were injected with K3K (daily, from the second day of infection, for 14 days at a dose of 10 mg/kg, and six, placebo (0.9% sodium chloride). After infection of the rabbits, HSV1 was monitored daily for cornea keratoconjunctivitis, encephalic disorders, and the presence of HSV1 antigens in peripheral blood lymphocytes using real-time PLR before and after infection. Prior to infection, all animals did not have the DNA of the virus in their blood, which indicated the absence of type 1 herpes virus in the peripheral blood. On the 3rd day after infection, HSV1 DNA was quantified in all animals in the blood, which amounted to 5.7*10⁶ copies of genomes/mL. In addition, three rabbits (two from the experimental group before treatment and one from the control group) developed encephal manifestations—convulsive syndrome, lack of appetite. All animals developed keratoconjunctivitis. On the 4th day after infection, the experimental group of rabbits was injected with KR at a dose of 10 mg/kg body weight into the ear vein, and a 0.9% sodium chloride solution was administered to the control group.

Every day for two weeks this procedure was repeated once a day. In the experimental group, all animals survived, and HSV1 DNA in the blood was not detected on days 13-14. In addition, in the experimental group, encephal manifestations disappeared by the 7th day of drug administration, while in the control 2 animals died. By the 14th day of treatment, one animal died in the experimental group, while in the control—6. Accordingly, the efficacy index was 83.3%, which indicates the high therapeutic efficacy of K3K in the model of herpetic keratoconjunctivitis/encephalitis in rabbits. In addition, the rabbits in the experimental group gained weight and all animals showed no signs of keratoconjunctivitis. The chemotherapeutic index for rabbits for K3K was 1000, which indicates the promise of K3K as a highly effective antiviral drug with a wide spectrum of action and low toxicity.

### Example 15. The Effect of K3K on the Humoral Immune Response to T-Dependent Antigen in Mice

Composition K3K is presented as an example of biological activity for a group of related derivatives provided for by the current application.

To investigate the effect of K3K, inbred mouse SPFs (Balb/cAωNCrl, 78 weeks old) were immunized with KLH, a T cell dependent antigen. 3 mice from the group were injected subcutaneously in the presence of Freund's complete adjuvant (50/50 v/v). A mixture of antigen (20 mg in 100 ml) with adjuvant (Sigma, #F5881) was emulsified and introduced into the neck. On the same day, 20 mg of K3K immunomodulator in 200 ml of PBS was administered intraperitoneally. Blood samples (5070 ml) were taken from mice on 7, 14, 21, and 28 days from a leg vein. Serum was prepared by coagulation of blood for 2 hours at 37° C., followed by 18 hours at 8° C., and centrifugation at 10,000 rpm in an Eppendorf-like centrifuge.

Serum was stored dissolved with an antibody stabilizer (SkyTec ABB500) at 4° C., and at the same time was analyzed by enzyme-linked immunosorbent assay ELISA. For the second sample, KLH (soluble, Sigma H7017) in phosphate buffered saline (PBS), 0.2 mg per well overnight at 4° C. was applied to 96-well plates for ELISA. Dissolved sera were incubated with antigen (200 mg per well) for 1 hour at room temperature, followed by washing the cells with PBS/0.1% Tween20. The binding of mouse antibodies to KLH was determined using isotope-specific anti-mouse immunoglobulins conjugated to HRP (Southern Biotechnology Ltd., anti-mouse IgM #102105, anti-mouse IgG1 #107005, anti-mouse IgG2a #108005, anti-mouse IgG2b #109005). TMB was used as a substrate.

The results were analyzed on a BioRad Photometer for a Model 550 microplate; optical density was measured at 595 nm. The titers of the used sera are from 1/300 to 1/20,000 in ½ increments (indicated on the X axis as 1 to 6, respectively). Serum reactivity is presented as O.D. shown by the sample in an ELISA. The dots represent the average reactivity of samples from 3 sera (from 3 mice provided). The spread of the factor represents a 95% confidence interval. After a single injection, the titer of a specific antibody on day 28 is significantly different between mice immunized with and without an immunomodulator. Thus, the titer of specific IgG1 in the sera of mice immunized in the presence of K3K was approximately 16 times higher, and the titer of IgG2a and IgG2b was 4 times higher than in control mice immunized with a single antigen.

The results were analyzed on a BioRad Photometer for a Model 550 microplate; optical density was measured at 595 nm. The titers of the used sera are from 1/300 to 1/20,000 in ½ increments (indicated on the X axis as 1 to 6, respectively). Serum reactivity is presented as O.D. shown by the sample in an ELISA. The dots represent the average reactivity of samples from 3 sera (from 3 mice provided). The spread of the factor represents a 95% confidence interval. After a single injection, the titer of a specific antibody on day 28 is significantly different between mice immunized with and without an immunomodulator. Thus, the titer of specific IgG1 in the sera of mice immunized in the presence of K3K was approximately 16 times higher, and the titer of IgG2a and IgG2b was 4 times higher than in control mice immunized with a single antigen.

### Example 16. The Effect of K3K on Gene Expression in Mouse Splenocytes, Determined Using PCR Matrix

Inbred SPF Balb/c mice (females, 12 weeks old) were given either antigen or K3K, or a combination of both. Injections were performed subcutaneously in the neck with an insulin needle. Only PBS was administered to control mice.

For antigen injection: 250 ml of a suspension of sterile lamb erythrocyte (SRBC from Quad Five inc., Cat #643100) was administered intraperitoneally through abdomen in the lateral side

The suspension was prepared as 2 ml of the initial suspension, washed 2 times (1500 rpm, 5 min) with PBS and resuspended in 2 ml. 10 ml of a 50% suspension was dissolved in 250 ml of PBS and introduced. 48 hours later, the mice were blocked, their spleen was isolated and placed in an RNALater (Ambion Inc, Cat #7021) immediately after isolation. Samples in RNALater were immediately frozen at 70° C. and maintained at this temperature until RNA was isolated. RNA isolation and PCR analysis of the matrix were performed as a service using SuperArray Inc according to their established protocol (www.superarray.com). Results. It was found that changes in mRNA expression based on PCR data are statistically significant if the difference with the control expression level was more than 3 times (or increase or decrease). From an expression analysis for 84 genes, the level of 75-85% of the genes in all samples was not statistically different from the control sample (mouse spleen injected with PBS instead of both antigen and immunomodulator (not shown). It is clear that a statistically significant difference was observed for a number of cytokine and chemokine genes and the corresponding receptors IL4, IL11, Spp1, IL10RA and to a lesser extent IL1f6, IL13, IL17b, IL20, IL6 and IL1R1).

Therefore, combinatorial compositions based on K3 have an activating effect on both humoral and cellular immunity and can be used as immunomodulators in various immunodeficiencies.

### Example 17. Various Pharmaceutical Compositions

Various methods of introducing supramolecular combinatorial polysaccharide derivatives (CPD) can be used. The CPD composition can be given orally or can be administered by intravascular, subcutaneous, intraperitoneal injection, in the form of an aerosol, by ocular route of administration, into the bladder, topically, and so on. For example, inhalation methods are well known in the art. The dose of the therapeutic composition will vary widely depending on the particular CPD administered, the nature of the disease, frequency of administration, route of administration, clearance of the agent used from the host, and the like. The initial dose may be higher with subsequent lower maintenance doses. The dose can be administered with a frequency of once a week or once every two weeks, or divided into smaller doses and administered once or several times a day, twice a week, and so on to maintain an effective dose level. In many cases, a higher dose will be needed for oral administration than for intravenous administration.

The compounds of this invention may be included in a variety of compositions for therapeutic administration. More specifically, the compounds of the present invention can be incorporated into pharmaceutical compositions in combination with suitable pharmaceutically acceptable carriers or diluents. Additionally, they can be incorporated into solid, semi-solid, liquid or gaseous forms, such as capsules, powders, granules, ointments, creams, foams, solutions, suppositories, injections, inhalation forms, gels, microspheres, lotions and aerosols. As such, the administration of the compounds can be carried out in various ways, including oral, buccal, rectal, parenteral, intraperitoneal, intradermal, transdermal, intratracheal administration and so on. The CPD of the invention can be distributed systemically after administration or can be localized using an implant or other composition that holds the active dose at the site of implantation. The compounds of the present invention can be administered alone, in combination with each other, or they can be used in combination with other known compounds (eg, perforin, anti-inflammatory agents, and so on). In pharmaceutical dosage forms, the compounds may be administered in the form of their pharmaceutically acceptable salts.

The following methods and excipients are given as examples only and are in no way limiting. For preparations for oral administration, the compounds can be used alone or in combination with suitable additives for the manufacture of tablets, powders, granules or capsules, for example, with conventional additives such as lactose, mannitol, corn starch or potato starch; with binding agents, such as crystalline cellulose, cellulose derivatives, gum arabic, corn starch or gelatins; with disintegrants such as corn starch, potato starch or sodium carboxymethyl cellulose; with mazyvayuschimi agents such as talc or magnesium stearate, and, if desired, with diluents, buffering agents, moistening agents, preservatives and flavoring agents.

The compounds can be incorporated into injectable compositions by dissolving, suspending or emulsifying them in an aqueous or non-aqueous solvent such as vegetable or other similar oils, synthetic aliphatic acid glycerides, esters of higher aliphatic acids or propylene glycol. Additionally, if desired, with conventional additives, such as solubilizers, isotonic agents, suspending agents, emulsifiers, stabilizers and preservatives. The compounds may be used in an aerosol composition for inhalation administration. The compounds of the present invention can be incorporated into suitable pressure propellants such as dichlorodifluoromethane, propane, nitrogen and the like. In addition, the compounds can be incorporated into suppositories by mixing with a variety of bases, such as emulsifying bases or water-soluble bases. The compounds of the present invention can be administered rectally using a suppository.

A suppository may contain excipients, such as cocoa butter, carbowax, and polyethylene glycols, which melt at body temperature but are solid at room temperature. Standard dosage forms for oral or rectal administration, such as syrups, elixirs and suspensions, where each unit dose, for example, a teaspoon, tablespoon, tablet or suppository, may contain a predetermined amount of a composition containing one or more compounds of the present invention. Similarly, unit dosage forms for injection or intravenous administration may contain the compound of the present invention in the composition in the form of a solution in sterile water, normal saline, or another pharmaceutically acceptable carrier. Implants for the sustained release of compositions are well known in the art.

Implants are made in the form of microspheres, plates, and so on with biodegradable or non-biodegradable polymers. For example, lactic and/or glycolic acid polymers form a degradable polymer that is well tolerated by the host. An implant containing a CPD according to the invention is positioned close to the site of infection, so that the local concentration of the active agent is increased compared to other areas of the body. As used herein, the term “unit dosage form” refers to physically discrete units suitable for use as single doses for human and animal subjects, each unit containing a predetermined number of compounds of the present invention, which, according to calculations, is sufficient to provide the desired effect together with a pharmaceutically acceptable diluent, carrier or excipient.

The descriptions of unit dosage forms of the present invention depend on the particular compound used, and the effect to be achieved, as well as the pharmacodynamics of the compound used in the host. Pharmaceutically acceptable excipients, such as excipients, adjuvants, carriers or diluents, are generally available. In addition, pharmaceutically acceptable excipients are generally available, such as pH adjusting agents and buffering agents, tonicity agents, stabilizers, wetting agents and the like. Typical doses for systemic administration range from 0.1 pg to 100 milligrams per kg of subject body weight per administration. A typical dose may be one tablet for administration from two to six times a day, or one capsule, or a sustained release tablet for administration once a day with a proportionally higher content of the active ingredient.

The effect of prolonged release may be due to the materials of which the capsule is made, dissolving at different pH values, capsules providing a slow release under the influence of osmotic pressure or any other known controlled release method. It will be clear to those skilled in the art that dose levels may vary depending on the particular compound, the severity of the symptoms, and the subject's predisposition to side effects. Some of the specific compounds are more potent than others. Preferred doses of this compound can be readily determined by those skilled in the art in a variety of ways.

A preferred method is to measure the physiological activity of the compound. One of the methods of interest is the use of liposomes as a vehicle for delivery. Liposomes fuse with the cells of the target region and ensure the delivery of liposome contents into the cells. The contact of the liposomes with the cells is maintained for a time sufficient for fusion using various methods of maintaining contact, such as isolation, binding agents and the like. In one aspect of the invention, liposomes are designed to produce an aerosol for pulmonary administration. Liposomes can be made with purified proteins or peptides that mediate membrane fusion, such as Sendai virus or influenza virus and so on. Lipids can be any useful combination of known liposome forming lipids, including cationic or zwitterionic lipids, such as phosphatidylcholine.

The remaining lipids will usually be neutral or acidic lipids, such as cholesterol, phosphatidylserine, phosphatidylglycerol and the like. To obtain liposomes, the method described by Kato et al. (1991) J. Biol. Chem. 266: 3361. Briefly, lipids and a composition for incorporation into liposomes containing CPP are mixed in a suitable aqueous medium, suitably in a salt medium, where the total solids content will be in the range of about 110 wt. %. After vigorous stirring for short periods of approximately 5-60 seconds, the tube is placed in a warm water bath at approximately 25-40° C. and this cycle is repeated approximately 5-10 times. The composition is then sonicated for a suitable period of time, typically approximately 1-10 seconds, and optionally further mixed with a vortex mixer. Then the volume is increased by adding an aqueous medium, usually increasing the volume by about 1-2 times, followed by agitation and cooling. The method allows to include supramolecular structures with high total molecular weight in liposomes.

Compositions with Other Active Agents

For use in the methods under consideration, the CPD of the invention can be included in compositions with other pharmaceutically active agents, in particular other antimicrobial, antiviral, hemostatic, activating regeneration agents, including pantothenic acid, cyanocobalamin, and cholecalciferol. Other agents of interest also include a wide range of antibiotics known in the art. Classes of antibiotics include penicillins, for example, penicillin G, penicillin V, methicillin, oxacillin, carbenicillin, nafcillin, ampicillin and so on; penicillins in combination with beta-lactamase inhibitors; cephalosporins, for example, cefaclorme, cefazalimine, cefazolemine, cefazolemine, cefazolemine, cefazolemine, monobactams; aminoglycosides; tetracyclines; macrolides; lincomycins; polymyxins; sulfonamides; quinolones; chloramphenicol; metronidazole; spectinomycin; trimethoprim; vancomycin; and so on. Antifungal agents are also useful, including polyenes, for example, amphotericin B, nystatin, flucosin; and azoles, for example miconazole, ketoconazole, itraconazole and fluconazole.

Anti-TB drugs include isoniazid, ethambutol, streptomycin and rifampin. Other agents of interest include a wide range of antiviral derivatives of mononucleotides and other RNA polymerase inhibitors known in the art. Classes of antiviral agents include interferons, lamivudine, ribavirin, etc. Other groups of antiviral agents include adefovir, vbacavir, didanosine, emtricitabine, lamivudine, stavudine, tenofovir, efavirenz, nevirapine, indinavir, lopinavir ritonavir, nelfinavir, ritonavir, sakinavir, daclatasvir, and Sovof. Cytokines, for example, interferon gamma, tumor necrosis factor alpha, interleukin 12, and so on, may also be included in the CPT composition of the invention. Above, the present invention is described by examples, which should not be construed as limiting the scope of the invention.

1. Biologically active combinatorial derivatives of polysaccharides, wherein a combinatorial derivative of polysaccharides is the supramolecular undivided combinatorial mixture of substituted polysaccharide derivatives with a maximum number of combinations is obtained, by simultaneous combinatorial modification of the polysaccharide with at least two covalent modifiers.

2. The invention according to p. 1., wherein the molar ratio of polysaccharide to covalent modifiers in the combinatorial synthesis reaction is calculated by the formulas:
k=n×(2ⁿ−1) (1)
m=4×(3×2ⁿ⁻²−1), (2)
Where
n=the number of groups available for substitution in the polysaccharide (in terms of monomer, a derivative of glucopyranose);
m=number of moles of the starting polysaccharide and the number of different molecules of combinatorial derivatives after synthesis (in terms of the glucopyranose derivative);
k=the number of moles of each of the two modifiers in the combinatorial synthesis reaction to obtain the maximum number of different derivatives;

3. A pharmaceutical composition containing biologically active combinatorial derivatives of polysaccharides according to claim 1, wherein it further comprises a lysine amino acid base.

4. The invention according to claim 1, wherein cellulose is used as the starting polysaccharide.

5. The invention according to claim 1, wherein carboxymethyl cellulose is used as the starting polysaccharide.

6. The invention according to claim 1, wherein carboxymethylpropyl cellulose is used as the starting polysaccharide.

7. The invention according to claim 1, wherein heparin is used as the starting polysaccharide.

8. The invention according to claim 1, wherein chitosan is used as the starting polysaccharide.

9. The invention according to claim 1, wherein succinylchitosan is used as the starting polysaccharide.

10. The invention according to claim 1, wherein carboxymethylchitosan is used as the starting polysaccharide.

11. The invention according to claim 1, wherein starch is used as the starting polysaccharide.

12. The invention according to claim 1, wherein carboxymethyl starch is used as the starting polysaccharide.

13. The invention according to claim 1, wherein methyl starch is used as the starting polysaccharide.

14. The invention according to claim 1, wherein the whole combinatorial mixture of polysaccharide derivatives in the form of a supramolecular structure is included in the pharmaceutical composition in the form of a powder and is used as a means to stop bleeding

15. The invention according to claim 1, wherein the whole combinatorial mixture of polysaccharide derivatives in the form of a supramolecular structure is included in the pharmaceutical composition and is used as an immunomodulating agent.

16. The invention according to claim 1, wherein the whole combinatorial mixture is part of the pharmaceutical composition and is used as an antiviral agent.

17. The invention according to claim 1, wherein the whole combinatorial mixture of polysaccharide derivatives in the form of a supramolecular structure is part of the pharmaceutical composition and is used as a wound healing agent.
